(19) United States
(12) Patent Application Publication
(10) Pub. No.: US 2015/0291741 A1
(43) Pub. Date: Oct. 15, 2015

(54) POLYOLS FROM PROTEIN BIOMASS

(71) Applicants: Ramani Narayan, Okemos, MI (US); Daniel Graiver, Midland, MI (US); Elodie Hablot, Mangiennes (FR)

(72) Inventors: Ramani Narayan, Okemos, MI (US); Daniel Graiver, Midland, MI (US); Elodie Hablot, Mangiennes (FR)

(21) Appl. No.: 14/567,100

(22) Filed: Dec. 11, 2014

Related U.S. Application Data
(60) Provisional application No. 61/916,534, filed on Dec. 16, 2013.

(51) Int. Cl. C08H 1/00; C07K 1/107; C08J 9/00; C07K 14/415

(52) U.S. Cl. CPC C08H 1/00; C07K 14/415; C07K 1/1077; C08J 9/00; C08J 2389/00

(57) ABSTRACT

A process of producing multi-hydroxy polyols, the process comprising providing a biomass material and transamidating the biomass material with an amine selected from the group consisting essentially of a diamine and an alkanolamine, to provide low molecular weight derived amino-acids or derived oligomers of proteins. Thereafter, reacting the derived amino-acids or derived oligomers with a carbonate to provide hydroxyl terminated urethane oligomers.

This application claims priority from U.S. Provisional application Ser. No. 61/916,534, filed Dec. 16, 2013.

### BACKGROUND OF THE DISCLOSURE

This disclosure is targeted towards utilizing the meal (protein-carbohydrate) residue remaining after extraction of the oil from soy 15 meal and other vegetable seeds or algae to produce polyols for the production of polyurethane foams.

Applicants are not aware of any published information on the process or compositions.

### BRIEF DESCRIPTION OF THE DRAWINGS

FIG. 1 shows an example of trans-amidation reaction of a protein with ethanol amine.

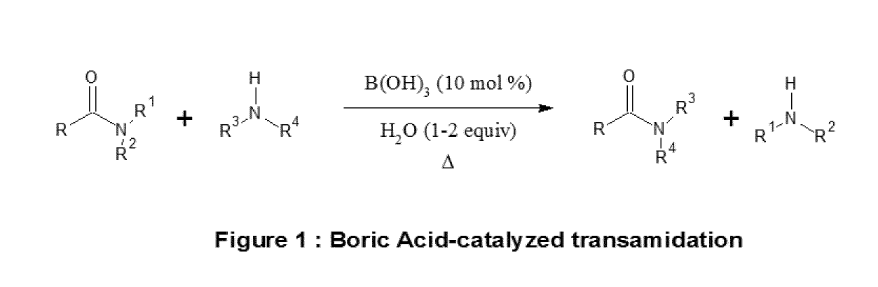

### THE DISCLOSURE

What is disclosed is a process of producing multi-hydroxy polyols, the process comprising providing a biomass material and transamidating the biomass material with a material selected from the group consisting essentially of diamine, triamine, multi-amine and alkanolamine compounds to provide low molecular weight derived amino-acids or derived oligomers of proteins. Thereafter, reacting the derived amino-acids or derived oligomers of proteins with a carbonate to provide hydroxyl terminated urethane oligomers.

Such polyols are useful in the production of polyurethanes, polyesters and polyacetals. Previously, biobased polyols were prepared from soybean oil, which is composed of triglycerides containing fatty acids. The unsaturated fatty acids are the precursors for the production of the polyols by adding hydroxyl functionality to the double bonds.

It is desirable that the hydroxyl functionality be primary rather than secondary in order to enhance the polyol reactivity. Thus, one major disadvantage of soybean oil is the presence of about 16% saturated fatty acids that are not reacted to produce the hydroxyl functionality.

In another embodiment, there is a process for producing multi-hydroxy polyols, the process comprising providing a material selected from a group consisting essentially of proteins, amino acids derived from proteins, and, mixtures of proteins and amino acids derived from proteins. Thereafter, transamidating any amino acids in said proteins or amino acids derived from said proteins with said amine or the alkanolamine or a diol to produce amino and hydroxyl-terminated monomers. Thereafter, reacting said monomers with a carbonate to provide hydroxyl terminated urethane oligomers.

Also contemplated within the scope of this invention are the compositions produced by the processes set forth Supra, and polyurethane products produced by the compositions produced by the processes set forth Supra.

### BACKGROUND OF THE DISCLOSURE

This disclosure is targeted towards utilizing the meal (protein-carbohydrate) residue remaining after extraction of the oil from soy meal and other vegetable seeds or algae to produce polyols for the production of polyurethane resins and foams. Applicants are not aware of any published information on the process or compositions.

In a typical process, the soybeans are cleaned and cracked to remove the hull. The hulls are lignocellulosic fibers and find industrial uses as reinforcing fillers or fibers in thermoplastic or thermoset matrices to provide biobased composites. The de-hulled bean is extracted with hexane solvent to give oil (18%) and a carbohydrate-protein residue (68%) called soy meal.

Polyols from the soy oil are known and are commonly prepared by glycerolysis. However, this procedure requires a reaction time of 5.5 hours at 220-240° C. and leads to breakdown of the triglyceride. It further promotes undesirable transesterification. The resulting product is inherently a mixture of hydroxyl esters, which again makes reproducibility an issue. Another common preparation is based on epoxidation of the unsaturated bonds of the fatty acids of the triglyceride in the soy oil followed by ring opening of the epoxides to yield secondary alcohols.

However side-reactions including transesterification, cyclization, polymerization and undesirable by-products also occur. Furthermore, due to incomplete epoxidation, residual double bonds remain and adversely affect the stability of resins made with these polyols. More importantly, secondary alcohols and alkoxides are obtained by this process. These secondary alcohols are significantly less reactive compared to primary alcohols. Thus, lack of reactivity and insufficient concentrations of polyols (low alcohol numbers) require blending soy-based polyol with conventional, petroleum-based, polyols.

The effort herein has recently developed and proposes a totally new chemical synthesis for the products. This consists of transamidifying soymeal proteins with a short amine chain to create amine intermediates, which can further react with a carbonate such as ethylene carbonate, to create a final polyol. This new chemical effort presents several advantages: primary (reactive) polyols are obtained, the resulting polyols already contain urethane linkages (carboxyamide groups).

The process is economical and does not require additional water (than that already present in soymeal) is added to the system. Furthermore, no waste or by-products are generated in the process, and all reagents, including the catalyst, are non-toxic and environmentally friendly materials.

The carboxyamide between the amino acids in the proteins is known to be chemically robust and it generally requires harsh conditions or highly evolved enzymes to react. Several catalysts are known to cleave this chemical bond by transamidation with amines. One example of such catalysts is NH₂OH₃.HCl, however, in order to be effective this catalyst must be used in relatively large quantities (up to 50 mol %).

The preferred catalyst however is the readily available, low-cost, nontoxic, and environmentally friendly boric acid. This catalyst is well known as a transamidation catalyst of amides and phthalimide with amines as previously reported by Ngyuen et al. (Organic Letters 2012, 14, (12), 3202).

It is reasonable to expect that polyols containing only primary hydroxyls should lead to better polyurethanes than those derived from secondary polyols. The higher reactivity of the primary soy-based polyols should also result in higher concentrations of soy-based reactant in the polyurethane and polyester formulations. Furthermore, since the resulting polyols contain multiple hydroxyl groups (e.g. high hydroxyl value), the process is particularly advantageous for the preparation of polyurethane resins and rigid foams.

### BRIEF SUMMARY

The present disclosure deals with polyol compositions comprising polyol compounds and the method of preparation of such compositions. These polyol compositions are derived from cleaving the amide bonds between the amino acids in proteins by transamidation reactions with amines and then reacting the intermediates with carbonate to obtain terminal, primary hydroxyls. Such polyol compositions can be used in polyurethane gel and foam compositions.

The protein derived polyol compositions in accordance with the present process comprise a reaction product of:

(a). A protein from vegetable, algae or animal sources comprising amino acids linked together by amide linkages to peptides and proteins. Common suitable proteins are those found in vegetable seeds after extracting the oil and removing the hulls, such as soy, jatropha and canola, or algae proteins after extracting oil for biodiesel.

(b). Amine compounds having multiple amine groups having the general formula:

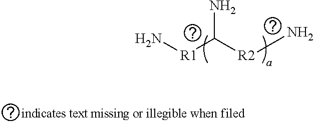

Where R1 and R2 are hydrocarbons, ethers or siloxanes consisting of 1-6 atoms and a is an integer of 0 or 1. Suitable amines can also be aromatic or cyclic compounds containing more than one amine functional group.

(c). a carbonate group such as cyclic ethylene or propylene carbonate, glycerol carbonate and similar carbonate compounds that react with amines to yield urethane bonds.

(d). a catalyst derived from boric acid that is known to affect the transamidation reaction of polyamides and polyimides with amines.

The present disclosure deals with a method of making polyurethane resins by contacting polyisocyanates with a polyol composition containing the present polyols, and other reactants, under suitable conditions sufficient to produce polyurethane resins and foams.

In another aspect, the polyol mixtures contain tertiary amines and imines that are used to catalyze the polyurethane reaction. Since these catalytic groups are chemically bound to the polyols the resultant products are substantially free of volatile amine emissions, yet the presence of multiple tertiary amine and imine groups provides an autocatalytic effect during the polyurethane gel and foam reactions.

The use of these polyols is beneficial as they limit the volatility of common amine catalysts and reduce the amount of volatile organic compounds (VOC). Thus they reduce workers exposures, lessen amines odors, improve safety, and address quality concerns. Thus,

[symbol]

### DETAILED DESCRIPTION

In accordance with the present disclosure, the transamidation methods of the proteins with suitable amines and subsequent reactions to produce the polyols are disclosed. A general method for producing these polyols comprises adding a protein biomass such as soymeal, excess ethylene diamine and boric acid to a reactor then flushing the reactor with nitrogen to prevent undesirable oxidation of the amines and heating the mixture to 150° C. for 6 hours. Excess ethylene diamine is then stripped out of the reactor, the temperature is adjusted to less than 80° C. and appropriate amounts of cyclic ethylene carbonate is added to convert the amines to the desired primary polyols as shown below:

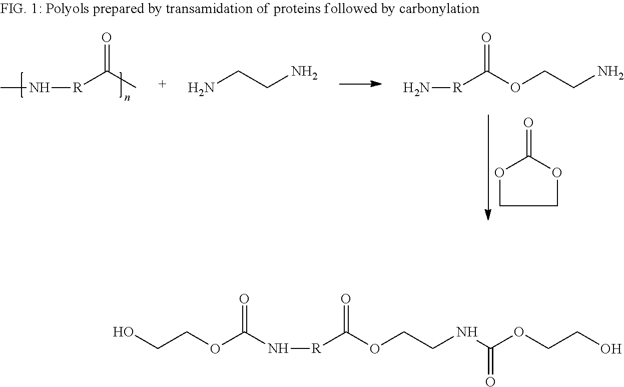

wherein R denotes an amino acid or a peptide that is composed of several amino acids linked by amide bonds.

Primary polyol compositions of the present disclosure as described are hydroxyl functional compounds having multiple primary hydroxyl moieties. The term “polyol” throughout this disclosure is used to encompass all such compounds having more than one hydroxyl moiety in their structure. Furthermore, polymeric polyol compositions also contain amines and imines that act as autocatalytic polyols when used in the reactions to yield polyurethane gels and foams.

These autocatalytic functional groups chemically bound to the polyols can either eliminate or reduce the need to include conventional urethane catalysts when formulating polyurethane resins and foams. However, conventional urethane catalysts can still be employed in the compositions or formulations along with the disclosed polyols since it is beneficial to balance the different reactions that occur in this process so resins and foams with acceptable properties may be produced.

The general procedure for producing the polyol composition from protein biomass is as follows. First, the protein is reacted with excess of the amine reagent containing multi amine groups. For illustration, the synthesis is described above for ethylene diamine, however, other reagents containing multiple amine groups can be used as well as ethanol amine and similar compounds. It is important to note that excess amine reagent is used in this synthesis as it is used instead of a solvent where the solid proteins are initially suspended in it and after the transamidation reaction is completed. The amine intermediates are dissolved in the amine reagent. It is also important to note that excess amine reagent must be used in order to suppress any polymerization reactions that will lead to high viscosity products.

Usually a temperature above 100° C. is necessary to drive the reaction to completion and the reaction time is inversely proportional to the reaction temperature. However, excessive temperature above 175° C. should not be used for prolonged periods of time as this will cause excessive thermal degradation.

Upon completion of the transamidation reaction, excess amine reagent that has not been reacted can be removed from the reaction mixture by adjusting the temperature and optional vacuum above the boiling temperature of the amine reagent and stripping it out of the reactor. The temperature of the reaction mixture is then adjusted to below 100° C. and the carbonate reagent is added slowly. It is important to add the carbonate slowly since its reaction with amines is mildly exothermic and unless care is taken the temperature could increase too fast leading to thermal degradation of the product. It is important to add the carbonate in a stoichiometric amount with respect to the amine. Insufficient carbonate will result in a product that contains residual amines which will lead to urea linkages when reacted with isocyanates to produce polyurethane.

There is no need for further post operations such as distillation to remove unreacted reagent or volatile materials, neutralization or purification of the product since the disclosed process does not lead to any waste or by-products that need to be removed and disposed of. However, vacuum distillation may be used to remove water if the initial protein biomass is not completely dry.

In accordance with the disclosed method of preparation, the polyol compositions do not contain volatile amines and therefore products derived from these polyols are substantially free of undesirable amine odors.

Applicants disclose several types of reagents and ranges in the present invention. These include, but are not limited to a range of different amines and different carbonates with a range of molecular weight to yield polyols with different hydroxyl numbers. Furthermore, a range of temperatures and reaction times are included which depends on the type of proteins and the desired viscosity and hydroxyl value of the polyol products.

The polyol compositions of the present invention are inherently a mixture of different derivatives of amino acids having different size molecules and different functionality. This polyol mixture is primarily defined by the original protein biomass used as a starting material. These polyols are useful for preparation of polyurethanes, polyesters or polyacetals and need not be exclusively made with one particular polyol. Such formulation can be comprised of different types of polyols composed of polyether polyol or polyester polyol or a combination thereof. The polyol formulation can consist of different ratios of polyols as determined by a formulator to achieve certain physical properties of the final polymer. Examples of polyether polyols include poly(alkyleneoxide) polymers such as poly(ethyleneoxide) and poly(propylene oxide) polymers and their copolymers with terminal hydroxyl groups derived from anhydric compounds, including diols and triols. Such polyols include, but are not limited to, ethylene glycol, propylene glycol, 1,3-butane diol, 1,6-hexane diol, neopentyl glycol, pentaerythritol, glycerol, diglycerol, trimethylolpropane, sugars and propoxylated sugars or combination thereof.

Examples of polyester polyols include those produced when a carboxylic acid is reacted with an excess of diol. Non-limiting examples include adipic acid or phthalic acid as well as phthalic anhydride reacting with ethylene glycol or butanediol. Other polyester polyols useful in the present invention are these produced by reacting a lactone with an excess of diol. For example, caprolactone reacted with propylene glycol.

These formulations can further include other additives that affect the polymerization process as well as the physical properties of the final product. For example, in the manufacture of polyurethanes the following additives can be included: water, blowing agents, cell stabilizing surfactants, co-catalysts, crosslinking agents, flame retardants, chain extenders, fillers, fibers, dyes, pigments and the like, or any combination thereof.

It is understood that other mixtures or materials that are known in the art can be included in the compositions and are within the scope of the present disclosure. Given the large number of components involved in the formulation of the end-products, there are different orders of combining these additives. However, a person skilled in the art would realize that the specific order of addition falls within the scope of the present disclosure.

### Example 1

### Polyols Obtained by Transamidation of Soymeal with Ethylene Diamine

Soymeal (50 gr.) ethylene diamine (300 gr.), water (50 gr.) and boric acid (16 gr.) were added to a 2 L Paar reactor. The reactor was then flushed with nitrogen and the reaction was allowed to proceed at 150° C. for 3 hr. Excess (unreacted) ethylene amine and water were then stripped out of the reactor and the temperature was decreased to 80° C. A sample of the product intermediates was collected and was titrated to determine the amine value (375 mgKOH/gr.) so that stoichiometric amounts of ethylene carbonate can be added. Accordingly, 20 gr. of amine derivative of the soymeal intermediate was added to a 100 mL round-bottomed flask and was heated to 80° C. under an inert atmosphere.

Then, 12.8 gr. (0.15 mole) ethylene carbonate was added slowly to maintain a temperature below 90° C. After all the ethylene carbonate was added, the reaction was allowed to continue for an additional 1 hr. in order to ensure complete reaction. The hydroxyl number of the product was measured by ASTM D 2083-92 and was 227 mg KOH/gr.

### Example 2

### Polyols Obtained by Non-Aqueous Transamidation of Soymeal with Ethylene Diamine

The same procedure as described in example 1 was repeated with no added water. Thus, Soymeal (50 gr.) ethylene diamine (300 gr.) and boric acid (16 gr.) were added to a 2 L Paar reactor. The reactor was then flushed with nitrogen and the reaction was allowed to proceed as before at 150° C. for 3 hr. Excess (unreacted) ethylene amine was stripped out of the reactor and the temperature was decreased to 80° C.

A sample of the product intermediates was collected and was titrated to determine the amine value (385 mgKOH/gr.) so stoichiometric amounts of ethylene carbonate could be added. Accordingly, 20 gr. of amine derivative of the soymeal intermediate was added to a 100 mL round-bottomed flask and was heated to 80° C. under an inert atmosphere. Then, 13.0 gr. (0.16 mole) of ethylene carbonate was slowly added to maintain a temperature below 90° C. After all the ethylene carbonate was added, the reaction was allowed to continue for an additional 1 hr. in order to ensure complete reaction. The hydroxyl number of the product was measured by ASTM D 2083-92 and was 232 mg KOH/gr.

### Example 3

### Polyols Obtained by Transamidation of Soy Isolate with Ethanol Amine

Soy isolate (250 gr.), ethanolamine (670 gr.), boric acid (75 gr.) and water (250 gr.) were added to a 2 L-Paar reactor. The reactor was flushed with nitrogen and the temperature was set to 150° C. The reaction was allowed to proceed at this temperature for 6 hr. and then the temperature was allowed to cool to 50° C. Water and excess ethanol amine were stripped under vacuum (200 Pa) from the reaction mixture. A sample of the product was collected and was titrated to determine the amine value (450 mgKOH/gr.) so stoichiometric amounts of ethylene carbonate could added. Accordingly, 20 gr. of amine derivative of the soymeal intermediate was added to a 100 mL round-bottomed flask and was heated to 80° C. under an inert atmosphere. Then, 13.0 gr. (0.16 mole) ethylene carbonate was added slowly to maintain a temperature below 90° C. After all the ethylene carbonate was added, the reaction was allowed to continue for an additional 1 hr. in order to ensure complete reaction. The hydroxyl number of the product was measured by ASTM D 2083-92 and was 235 mg KOH/gr.

The use of ethanolamine has several advantages over ethylene diamine as it introduces hydroxyl groups on the transamidated product, requiring less carbonate to be used in the final carbonylation step. Furthermore, the boiling point of ethanol amine is higher than ethylene diamine, which leads to a reduced pressure at the reaction temperature. Also, ethanolamine is less corrosive then ethylene diamine.

### Example 4

### Polyols Obtained by Non-Aqueous Transamidation of Soymeal with Ethanol Amine

Jatropha meal (250 gr.), ethanolamine (300 gr.) and boric acid (75 gr.) were introduced in a 2 L-Paar reactor. The reactor was flushed with nitrogen and the temperature was set to 150° C. The transamidation reaction was allowed to proceed at this temperature for 6 hr. and then the temperature was lowered to 50° C. Excess ethanol amine was stripped under vacuum (200 Pa) from the reaction mixture. A sample of the product was collected and was titrated to determine the amine value (306 mgKOH/gr.) so stoichiometric amounts of ethylene carbonate could be added. Accordingly, ethylene carbonate was slowly added to maintain a temperature below 60° C. After all the ethylene carbonate was added, the reaction was allowed to continue for additional 1 hr. in order to ensure complete reaction. The hydroxyl number of the product was measured by ASTM D 2083-92 and was 195 mg KOH/gr.

The present disclosure deals with a method of making polyurethane resins by contacting polyisocyanates with a polyol composition containing the present polyols, and other reactants, under suitable conditions sufficient to produce polyurethane resins and foams.

What is claimed is:

1. A process for producing multi-hydroxy polyols, said process comprising:
a. providing a biomass material;
b. transamidating said biomass material with a material selected from the group consisting essentially of
i. a diamine,
ii. triamine, or
ii. an alkanolamine,
to provide low molecular weight derived amino-acids or derived oligomers of proteins;
c. reacting said derived amino-acids or derived oligomers of protein with a carbonate to provide hydroxyl terminated urethane oligomers.

2. A process of producing multi-hydroxy polyols as claimed in claim 1 wherein, in addition, there is a step of alkoxylating any carbohydrates in the biomass to produce hydroxyl groups.

3. A product produced by the process of claim 1.

4. A product produced by the process of claim 2.

5. A process as claimed in claim 1 wherein the biomass material is a meal produced by the extraction of oil from vegetable seed.

6. A process of producing multi-hydroxy polyols, said process comprising:
a. providing a material selected from a group consisting essentially of i. proteins, ii. amino acids derived from proteins, and, iii. mixtures of i. and ii.;
b. transamidating any amino acids in said proteins or amino acids derived from said proteins with a material selected from the group consisting of diamine, triamine, an alkanolamine or a diol to produce amino and hydroxyl-terminated monomers;
c. reacting said monomers with a carbonate to provide hydroxyl terminated urethane oligomers.

7. A product produced by the process of claim 6.

8. A process of producing multi-hydroxy polyols as claimed in claim 6 wherein, in addition, there is a step d. of alkoxylating any carbohydrates in the protein to produce hydroxyl groups.

9. A product produced by the process of claim 8.

10. A polyurethane composition produced using the product claimed in claim 3.

11. A polyurethane composition produced using the product claimed in claim 4.

12. A polyurethane composition produced using the product claimed in claim 7.

13. A polyurethane composition produced using the product claimed in claim 9.

14. A polyurethane composition produced using the product claimed in claim 9.

15. A polyester composition produced using the product claimed in claim 3.

16. A polyester composition produced using the product claimed in claim 4.

17. A polyester composition produced using the product claimed in claim 7.

18. A polyester composition produced using the product claimed in claim 9.

19. A polyester composition produced using the product claimed in claim 9.

20. A polyacetal composition produced using the product claimed in claim 3.

21. A polyacetal composition produced using the product claimed in claim 4.

22. A polyacetal composition produced using the product claimed in claim 7.

23. A polyacetal composition produced using the product claimed in claim 9.

24. A polyacetal composition produced using the product claimed in claim 13.

25. The process as claimed in 1 wherein the carbonate is selected from the group consisting essentially of i. ethylene carbonate, ii. propylene carbonate, iii. butylene carbonate, iv. glycerol carbonate, and v. any combination of i. to iv.

26. A polyol produced by the process of claim 4 wherein the amino acid is a single, essentially pure amino acid.

27. The process as claimed in claim 1, wherein the molar ratio of the carbonate to the amines ranges between 1:10 to 10:1.

28. The process as claimed in claim 27, wherein the molar ratio of the carbonate to the amines ranges between 1:2 to 2:1.

29. A rigid polyurethane foam which has a foam density in the range from 15 to 65 g/l. when produced from the process as claimed in claim 1.

30. An insulating rigid foam produced using the product as claimed in claim 3.
